(19) United States
(12) Patent Application Publication
(10) Pub. No.: US 2019/0054032 A1
(43) Pub. Date: Feb. 21, 2019

(54) PHARMACEUTICAL COMPOSITION COMPRISIONG BILE SALT, PREPARATION METHOD THEREOF, AND APPLICATION OF SAME

(71) Applicant: HANGZHOU PUSH-KANG BIOTECHNOLOGY CO., LTD, Hangzhou (CN)

(72) Inventors: Xiaomin ZHANG, Hangzhou (CN); Yingxin ZHANG, Hangzhou (CN); Ju YAO, Hangzhou (CN); Bo YU, Hangzhou (CN)

(21) Appl. No.: 16/081,026

(22) PCT Filed: Mar. 2, 2017
(86) PCT No.: PCT/CN2017/075505

§ 371 Date: Aug. 29, 2018

(30) Foreign Application Priority Data
Mar. 14, 2016 (CN) 201610149312.8

(51) Int. Cl. A61K 9/51; A61K 31/337; A61P 35/00

(52) U.S. Cl. CPC A61K 9/5123; A61K 31/337; A61P 35/00; A61K 9/5192; A61K 9/5146

(57) ABSTRACT

There are provided a pharmaceutical composition, a preparation method thereof, and an application of same. The pharmaceutical composition includes an active ingredient, a polymer, and a surfactant. The surfactant includes a bile salt. The pharmaceutical composition is prepared as a nanoparticle.

### CROSS-REFERENCE TO RELATED APPLICATIONS

This application is a national stage application of PCT Application No. PCT/CN2017/075505. This Application claims priority from PCT Application No. PCT/CN2017/075505, filed Mar. 2, 2017, and CN Application No. 201610149312.8, filed Mar. 14, 2016, the contents of which are incorporated herein in the entirety by reference.

Some references, which may include patents, patent applications, and various publications, are cited and discussed in the description of the present disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### TECHNICAL FIELD

The present disclosure relates to the field of pharmaceutical formulations and, in particular, to a bile salt-containing pharmaceutical composition. The disclosure is also directed to a method of preparing the pharmaceutical composition and use thereof.

### BACKGROUND

Cancer is the top threat to human lives and health. Chemotherapy is the most important means of cancer treatment. However, most chemotherapeutic drugs are lack of specificity to target tumor tissues and cannot selectively kill tumor cells without harg healthy cells. In particular, for cancer cells with resistance to anticancer drugs, higher doses tend to be given to overcome the drug resistance. In such cases, healthy cells will be subject to greater side effects of the drugs and, sometimes, even the treatment has to be interrupted for this reason. Moreover, for most anti-tumor agents, their treatment efficacy is further limited by some of their own properties (e.g., poor water solubility, narrow therapeutic windows, etc.).

Since the 1970s, cancer treatment using nanocarriers as delivery systems for chemotherapeutic drugs has attracted great attention. Nanoparticles used in drug delivery systems can be made from a variety of materials such as polymers, lipids and organometallic complexes.

In recent years, polymer-based nano-formulations have become a focus of interest thanks to their good physical and chemical properties. Nanoparticles (NPs) are solid, colloidal particles formed of macromolecular substances and ranging in size from 10 nm to 1,000 nm. When dispersed in water, nanoparticles can form a quasi-colloidal solution. Due to the superiority of nanoparticles as drug carriers, these substances have become an important focus of medical and pharmaceutical research both in China and abroad.

Adjuvants used in nanoparticle formulations are mostly degradable macromolecular polymers, among which polyesters are the biodegradable macromolecular materials that have been most studied and most widely used up to now. Commonly-used polyesters are polylactic acid (PLA), polyglycolic acid (PGA), poly (lactic-co-glycolic acid) (PLGA) and polycaprolactone (PCL), etc.

PLA- and PLGA-based nanoparticles are usually surface-modified because, if not, they will be easily recognized and phagocytized by macrophages, which may shorten their circulation times in the body and make them not able to fully exert their pharmacological effects. Surface modifiers that are commonly used include, among other substances, polyethylene glycol (PEG), polyvinyl alcohol (PVA), povidone, heparin, human serum albu, sialic acids and gangliosides, with PEG being most commonly used.

Although there have been some conventional nanoparticles available for use, they are suffering from many deficiencies such as low encapsulation efficiency, rapid release, poor targeting performance and low in vivo efficacy. Therefore, there is still an urgent need in this art for new polymer-based nanoparticles with better characteristics.

### SUMMARY

In one aspect, the present application is directed to a pharmaceutical composition comprising an active ingredient, a polymer and a surfactant. The surfactant comprises a bile salt and the pharmaceutical composition comprises nanoparticles.

In certain embodiments, the active ingredient is a hydrophobic substance. In certain embodiments, the active ingredient is selected from an anti-neoplastic agent, an antibiotic agent, a cardiovascular agent, an anti-diabetic agent, a non-steroidal anti-inflammatory agent or a combination thereof. In certain embodiments, the active ingredient is selected from paclitaxel, camptothecin and a derivative thereof. In certain embodiments, the active ingredient is paclitaxel, docetaxel, cabazitaxel or hydroxycamptothecin.

In certain embodiments, the polymer is selected from PLGA, PLA, a PLGA or PLA derivative, or a combination thereof. In certain embodiments, the PLGA or PLA derivative is a polyethylene glycol (PEG) derivative of PLGA or PLA. In certain embodiments, the polymer is selected from PEG-PLA, PEG-PLGA or a combination thereof.

In certain embodiments, the bile salt is selected from sodium cholate, sodium deoxycholate, sodium taurocholate or a combination thereof.

In certain embodiments, the surfactant excludes lipid surfactants. Preferably, the surfactant excludes phospholipids.

In certain embodiments, the pharmaceutical composition is nanoparticles.

In certain embodiments, the polymer and the active ingredient are present in a ratio by weight ranging from 5:1 to 40:1.

In certain embodiments, the surfactant and the active ingredient are present in a ratio by weight ranging from 0.1:1 to 4:1.

In certain embodiments, the surfactant and the polymer are present in a ratio by weight ranging from 1:5 to 1:50.

In certain embodiments, the pharmaceutical composition has an average particle size in the range of 50-200 nm.

In another aspect, the present application is directed to a method for preparing the pharmaceutical composition hereof, comprising the steps of: (i) dissolving the polymer and the active ingredient in an organic solvent; (ii) dissolving the surfactant in water; (iii) mixing the aqueous solution from step (ii) with the organic solution from step (i) under the action of a shear force; and (iv) removing the organic solvent.

In certain embodiments, the organic solvent is selected from acetone, dichloromethane, acetonitrile or a combination thereof.

In certain embodiments, the shear force results from agitation.

In certain embodiments, step (iii) further includes adding the aqueous solution from step (ii) dropwise to the organic solution from step (i).

In certain embodiments, the organic solution and the aqueous solution are present in a ratio ranging from 1:10 to 20:1.

In certain embodiments, the surfactant is present at a concentration of from 0.05 mg/mL to 1 mg/mL in the aqueous solution.

In certain embodiments, the active ingredient is present at a concentration of from 0.1 mg/mL to 1 mg/mL in the organic solvent.

In certain embodiments, the polymer is present at a concentration of from 2 mg/mL to 10 mg/mL in the organic solvent.

In a further aspect, the present application is directed to the use of the pharmaceutical composition hereof in the preparation of a medicament for mitigating, treating, or preventing a disease.

In certain embodiments, the disease is cancer.

In a further aspect, the present application is directed to the use of the pharmaceutical composition hereof in the mitigation, treatment or prevention of a disease.

In certain embodiments, the disease is cancer.

In a further aspect, the present application is directed to a method for mitigating, treating, or preventing a disease, comprising applying an effective amount of the pharmaceutical composition hereof on a subject in need thereof.

In certain embodiments, the disease is cancer.

### BRIEF DESCRIPTION OF THE DRAWINGS

FIG. 1 is a diagram showing a particle size distribution of paclitaxel-loaded nanoparticles prepared in Example 1 and a TEM image of the paclitaxel-loaded nanoparticles.

FIG. 2 shows an image of a lyophilized powder of the paclitaxel-loaded nanoparticles prepared in Example 1 and an image of a solution resulting from reconstitution of the powder.

FIG. 3 is a diagram showing in vitro release profiles of Preparation I (paclitaxel-loaded nanoparticles prepared in Example 1), Preparation II (paclitaxel-loaded nanoparticles prepared in Example 4) and a commercially available paclitaxel injection in accordance with Example 8.

FIG. 4 is a diagram showing profiles of rat plasma concentration of paclitaxel over time from Preparation I (paclitaxel-loaded nanoparticles prepared in Example 1), Preparation II (paclitaxel-loaded nanoparticles prepared in Example 4) and a commercially available paclitaxel injection in accordance with Example 9.

FIG. 5 is a diagram showing in vivo distribution in tissues of Preparation I (paclitaxel-loaded nanoparticles prepared in Example 1), Preparation II (paclitaxel-loaded nanoparticles prepared in Example 4) and a commercially available paclitaxel injection in accordance with Example 10.

FIG. 6 is a diagram showing growth inhibition by Preparation I (paclitaxel-loaded nanoparticles prepared in Example 1), Preparation II (paclitaxel-loaded nanoparticles prepared in Example 4) of tumor xenografts in nude mice induced by BEL-7402 liver cancer cells and a commercially available paclitaxel injection and body weight variation thereof in accordance with Example 11.

### DETAILED DESCRIPTION

In one aspect of the present disclosure, there is provided a pharmaceutical composition comprising an active ingredient, a polymer and a surfactant. The surfactant comprises a bile salt and the pharmaceutical composition comprises nanoparticles.

### Active Ingredient

A person of skill in the art may properly select the active ingredient according to the practical need. In certain embodiments, the active ingredient is a hydrophobic substance.

As used herein, the term “hydrophobic substance” refers to a substance with a solubility of less than 1 g, 0.1 g, 0.01 g, 1 mg or 0.5 mg in 100 g of water at 25° C.

In certain embodiments, the active ingredient is selected from an anti-neoplastic agent, an antibiotic agent, a cardiovascular agent, an anti-diabetic agent, a non-steroidal anti-inflammatory agent or a combination thereof.

Illustrative examples of the active ingredient hereof may be: anti-neoplastic agents, such as paclitaxel, docetaxel, cabazitaxel, 5-fluorouracil, etoposide, phenylalanine mustard, chlorambucil, hexamethylmelae, methotrexate, methyl-CCNU, vinorelbine, teniposide, homoharringtonine, hydroxycamptothecin, etc.; antibiotic agents, such as chloramphenicol, erythromycin, erythromycin estolate, erythromycin ethylsuccinate, midecamycin, josamycin, clarithromycin, rokitamycin, sulfadiazine, trimethoprim, furantoin, rifampicin, rifaxi, rifandin, dapsone, acedapsone, miconazole, etc.; cardiovascular agents, such as nifedipine, nicardipine, nitrendipine, nilvadipine, cinnarizine, perhexiline, molsidoe, digitoxin, digoxin, lanatoside C, deslanoside, propafenone, amiodarone, nitroglycerin, pentaerithrityl tetranitrate, cyclandelate, tocopherol nicotinate, etc.; anti-diabetic agents, such as tolbutamide, glibenclamide, glipizide, etc.; and non-steroidal anti-inflammatory agents, such as clemastine, cyproheptadine, pizotifen, ketotifen, tranilast, etc. Reference can be made for the structures of the particular drugs disclosed above to the instructions thereof approved by drug adistrations in different countries or regions, for example, those approved by the China Food and Drug Adistration, U.S. Food and Drug Adistration, Japanese Pharmaceuticals and Medical Devices Agency or European Medicines Agency. In certain embodiments, the active ingredient is paclitaxel, camptothecin or a derivative thereof.

As used herein, the term “derivative” means a compound resulting from the replacement of an atom or a group of atoms in a parent compound molecule by another atom or group of atoms. Derivatives of paclitaxel include, but are not limited to, its derivatives with succinic and glutaric acids, sulfonates, derivatives with ao acids, phosphates, organic acid esters and carbonates, N-methyl pyridinium salts, and derivatives with polyethylene glycol, derivatives with polymethacrylic acid, and derivatives with polyglutamic acid or polyaspartic acid.

In certain embodiments, the active ingredient is paclitaxel, docetaxel, cabazitaxel (7β, 10β-dimethoxydocetaxel) or hydroxycamptothecin.

All the compounds described herein also include their salts, esters, mesomeric, racemic and isomeric forms. The isomers mentioned herein include both cis-trans and optical isomers.

### Polymer

A person of skill in the art may properly select the polymer according to the practical need. In certain embodiments, the polymer is a degradable macromolecular material. In certain embodiments, the polymer is selected from PLGA, PLA, a PLGA or PLA derivative or a combination thereof.

As used herein, the term “PLGA or PLA derivative” refers to a compound resulting from a modification to the basic structure of PLGA or PLA. The modification may include a group modification which changes its hydrophilic or hydrophobic properties.

In certain embodiments, the PLGA or PLA derivative is a polyethylene glycol (PEG) derivative of PLGA or PLA. In certain embodiments, the polymer is selected from PEG-PLA, PEG-PLGA or a combination thereof.

The composition and molecular weight range of the polymer used herein may be either commercially available or commonly used in drug delivery systems. In certain embodiments, the composition and molecular weight range of the polymer may be selected based on a target sustained release time.

In certain embodiments, the molecular weight range of the polymer used herein may be 0.5 K-500 K. In certain embodiments, the molecular weight range of the polymer used herein may be 0.5 K-300 K, 1 K-300 K, 3 K-300 K, 5 K-300 K, 8 K-300 K, 10 K-300 K, 12 K-300 K, 15 K-300 K, 18 K-300 K, 1 K-200 K, 5 K-150 K, 8 K-100 K, 10 K-50 K, 15 K-30 K or 18 K-25 K.

As mentioned herein, a molecular weight may either be a weight-average molecular weight or a number-average molecular weight. A method commonly used in the art may be employed for molecular weight deteration, such as light scattering, ultracentrifuge sedimentation or gel chromatography.

In certain embodiments, the polymer used herein may be either end-capped or not. In certain embodiments, the polymer used herein is a polymer end-capped with a methoxy, ethoxy, methacryloyl or acetyl group.

In certain embodiments, the PLGA used herein has a ratio of LA to GA in the range of 1:4-6:1, 1:3-6:1, 1:2-6:1, 1:1-6:1, 2:1-6:1, 3:1-6:1, 1:4-5:1, 1:4-4:1, 1:4-3:1, 1:2-4:1, 1:1-4:1 or 2:1-4:1. In certain embodiments, the ratio of LA to GA in the PLGA used herein is 50:50, 75:25 or 85:15.

### Bile Salt

Bile salts are major constituents of bile (a greenish-yellow fluid excreted by the liver). Human bile is rich in bile salts which play an important role in the absorption of lipids, fat-soluble vitas and drugs and are regarded as “physiological detergents”. Bile salts contain hydrophilic hydroxyl and carboxyl groups as well as hydrophobic methyl and “—CH₂—” groups, which impart to them interfacial activity and the ability to reduce the surface tension between lipid-water phases. Therefore, Bile salts can used to solubilize many sparingly soluble drugs.

In certain embodiments, the bile salt used herein is selected from sodium cholate, sodium deoxycholate, sodium taurocholate or a combination thereof.

In certain embodiments, the sodium cholate hereof has the following structure:

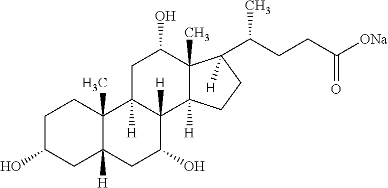

In certain embodiments, the sodium deoxycholate hereof has the following structure:

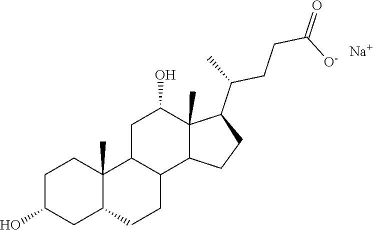

In certain embodiments, the sodium taurocholate hereof has the following structure:

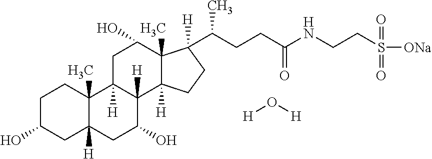

Without wishing to be bound by theory, the addition of the bile salt imparts to the prepared nanoparticles higher encapsulation efficiency, more excellent sustained-release performance, higher in vivo targeting performance and better in vivo efficacy.

### Surfactant

In certain embodiments, the pharmaceutical composition hereof includes another surfactant than the bile salt. In certain embodiments, the pharmaceutical composition hereof excludes lipid surfactants. In certain embodiments, the pharmaceutical composition hereof excludes phospholipids.

In certain embodiments, the pharmaceutical composition hereof does not include any other surfactant than the bile salt.

In certain embodiments, the surfactant hereof is not covalently bonded to the active ingredient.

In certain embodiments, the bile salt in the pharmaceutical composition hereof is not covalently bonded to the active ingredient.

### Composition Ratio

A person of skill in the art may select a ratio of the active ingredient to the polymer according to the practical need. In certain embodiments, the polymer and active ingredient are present in a ratio by weight ranging from 5:1 to 40:1. In certain embodiments, the polymer and the active ingredient are present in the composition in a ratio by weight in the range of 5:1-35:1, 5:1-30:1, 5:1-25:1, 5:1-23:1, 5:1-21:1, 6:1-35:1, 8:1-35:1, 10:1-35:1, 12:1-35:1, 15:1-35:1, 16:1-35:1, 18:1-35:1, 6:1-30:1, 8:1-28:1, 10:1-25:1, 12:1-24:1, 15:1-22:1 or 18:1-22:1.

A person of skill in the art may select a ratio of the surfactant to the active ingredient according to the practical need. In certain embodiments, the surfactant and the active ingredient are present in a ratio by weight ranging from 0.1:1 to 4:1. In certain embodiments, the surfactant and the active ingredient are present in the composition in a ratio by weight in the range of 0.2:1-4:1, 0.3:1-4:1, 0.4:1-4:1, 0.5:1-4:1, 0.6:1-4:1, 0.7:1-4:1, 0.8:1-4:1, 0.9:1-4:1, 1:1-4:1, 2:1-4:1, 3:1-4:1, 0.2:1-3:1, 0.2:1-2:1, 0.2:1-1:1, 0.2:1-0.8:1, 0.2:1-0.6:1, 0.2:1-0.5:1, 0.3:1-4:1, 0.4:1-3:1, 0.5:1-2:1 or 1:1-2:1.

A person of skill in the art may select a ratio of the surfactant to the polymer according to the practical need. In certain embodiments, the surfactant and the polymer are present in a ratio by weight ranging from 1:5 to 1:50. In certain embodiments, the surfactant and the polymer are present in the composition in a ratio by weight in the range of 1:5-1:45, 1:5-1:42, 1:5-1:40, 1:5-1:35, 1:5-1:30, 1:5-1:25, 1:5-1:20, 1:5-1:15, 1:5-1:10, 1:6-1:50, 1:7-1:50, 1:8-1:50, 1:9-1:50, 1:10-1:50, 1:12-1:50, 1:15-1:50, 1:18-1:50, 1:20-1:50, 1:25-1:50, 1:30-1:50, 1:35-1:50, 1:40-1:50, 1:6-1:45, 1:8-1:42, 1:10-1:40, 1:35-1:40, 1:10-1:15, 1:6-1:15 or 1:8-1:12.

### Composition

In certain embodiments, the composition hereof is a solid formulation. Examples of the solid formulation include tablets, capsules, granules, powders or lozenges. In certain embodiments, the composition is nanoparticles. In certain embodiments, the composition is dried nanoparticles. In certain embodiments, the composition is lyophilized nanoparticles.

In certain embodiments, the nanoparticles have a particle size of 10-500 nm. In certain embodiments, the particle size of the nanoparticles ranges from 50 nm to 200 nm. In certain embodiments, the particle size of the nanoparticles is in the range of 10-400 nm, 10-300 nm, 10-250 nm, 10-200 nm, 10-150 nm, 10-120 nm, 10-100 nm, 10 -90 nm, 20-90 nm, 30-90 nm, 40-90 nm, 50-90 nm, 60-90 nm, 70-90 nm, or 70-110 nm. In certain embodiments, the particle size of the nanoparticles ranges from 10 nm to 100 nm.

The particle size may be detered using a method commonly employed in the art, such as scanning electron microscopy (SEM) and light scattering. In certain embodiments, the particle size is detered by means of light scattering. In certain embodiments, the particle size is detered using a dynamic laser scattering instrument.

The nanoparticles hereof have an acceptable coefficient of dispersion. In certain embodiments, the coefficient of dispersion of the nanoparticles hereof is not greater than 0.3, 0.2, 0.19 or 0.18.

It will be appreciated by those skilled in the art that the composition hereof may be further modified. In certain embodiments, the composition hereof may be provided with a further encapsulation for, for example, sustained or controlled release. In certain embodiments, the composition hereof may be surface modified with targeting groups (e.g., antibodies, ligands, specific substrates, etc.) or other macromolecules for further improving the targeting properties or other kinetic parameters of the composition hereof, or for traceability of the composition hereof.

It will be appreciated by those skilled in the art that, the composition further comprises other pharmaceutically acceptable ingredients, apart from the active ingredient, the polymer and the surfactant. In certain embodiments, the other ingredients include a lyoprotectant including, but not limited to, lactose, mannose, dextran, sucrose and glycine. In certain embodiments, the other ingredients include a solution including, but not limited to, a sodium chloride solution, a glucose solution, a PBS buffer, an ethanol solution or the like.

As used herein, the term “pharmaceutically acceptable” refers to compounds, materials, compositions and/or formulations that are within the scope of proper medicinal assessment, suitable for use in contact with patient tissues, without undue toxicity, irritation, allergic response or other issues or complications, commensurate with a reasonable benefit/risk ratio and effective for the intended use.

The composition hereof is suitable to be adistered by any appropriate route, for example, orally (including buccally or sublingually), rectally, nasally, topically (including buccally, sublingually or transdermally), vaginally or parenterally (including by subcutaneous, intradermic, intramuscular, intra-articular, intra-synovial, intrasternal, intrathecal, intralesional, intravenous or subdermal injection or infusion). In certain embodiments, the composition hereof is adistered parenterally. In certain embodiments, the composition hereof is adistered by intravenous infusion. In certain embodiments, the composition hereof is adistered subcutaneously.

### Beneficial Effects

Without wishing to be bound by theory, the pharmaceutical composition hereof has one or more of the following advantages: 1) higher encapsulation efficiency; 2) a more uniform particle size distribution; 3) more excellent stability; 4) higher targeting performance; 5) improved penetration in tumors; 6) higher efficacy; and 7) a higher load of the active ingredient.

In another aspect of the present disclosure, there is provided a method for preparing the composition hereof, including the steps of: (i) dissolving the polymer and the active ingredient in an organic solvent; (ii) dissolving the surfactant in water; (iii) mixing the aqueous solution from step (ii) with the organic solution from step (i) under the action of a shear force; and (iv) removing the organic solvent.

### Step (i): Dissolution of Polymer and Active Ingredient in Organic Solvent

A person of skill in the art may properly select the organic solvent according to the solubility of the active ingredient and the requirements of the preparation process. In certain embodiments, the organic solvent is selected from acetone, dichloromethane, acetonitrile or a combination thereof. In certain embodiments, the organic solvent is acetone.

In certain embodiments, the active ingredient is present in the organic solvent at a concentration of 0.1-1 mg/mL. In certain embodiments, the active ingredient is present in the organic solvent at a concentration of 0.1-1 mg/mL, 0.2-1 mg/mL, 0.3-1 mg/mL, 0.4-1 mg/mL, 0.5-1 mg/mL, 0.6-1 mg/mL, 0.7-1 mg/mL, 0.8-1 mg/mL, 0.1-0.9 mg/mL, 0.1-0.8 mg/mL, 0.1-0.7 mg/mL, 0.1-0.6 mg/mL, 0.1-0.5 mg/mL, 0.1-0.4 mg/mL, 0.1-0.3 mg/mL, 0.2-0.6 mg/mL or 0.3-0.5 mg/mL.

In certain embodiments, the polymer is present in the organic solvent at a concentration of 2-10 mg/mL. In certain embodiments, the polymer is present in the organic solvent at a concentration of 2-9 mg/mL, 2-8 mg/mL, 2-7 mg/mL, 2-6 mg/mL, 2-5 mg/mL, 3-10 mg/mL, 3-9 mg/mL, 3-8 mg/mL, 3-7 mg/mL, 3-6 mg/mL, 3-5 mg/mL, 3-9 mg/mL or 4-8 mg/mL.

### Step (ii): Dissolution of Surfactant in Aqueous Solution

In certain embodiments, the surfactant is present in the aqueous solution at a concentration of 0.05-1 mg/mL. In certain embodiments, the surfactant is present in the aqueous solution at a concentration of 0.06-1 mg/mL, 0.07-1 mg/mL, 0.08-1 mg/mL, 0.09-1 mg/mL, 0.1-1 mg/mL, 0.2-1 mg/mL, 0.3-1 mg/mL, 0.05-0.9 mg/mL, 0.05-0.8 mg/mL, 0.05-0.7 mg/mL, 0.05-0.6 mg/mL, 0.05-0.5 mg/mL, 0.05-0.4 mg/mL, 0.06-0.8 mg/mL, 0.08-0.6 mg/mL, 0.08-0.5 mg/mL, 0.08-0.4 mg/mL or 0.1-0.3 mg/mL.

Step (iii): Mixing of Aqueous Solution from Step (ii) with Organic Solution from Step (i) Under Action of Shear Force

In certain embodiments, step (iii) further includes adding the aqueous solution from step (ii) dropwise to the organic solution from step (i).

According to the present application, the shear force may be provided by agitation, shearing or homogenization, provided that the shear force is not greater than a shear force generated by mechanical agitation at 1,000 rpm, 800 rpm, 700 rpm, 600 rpm, 500 rpm or 400 rpm. In certain embodiments, the shear force results from agitation. In certain embodiments, the shear force results from mechanical agitation. In certain embodiments, the agitation is performed at a speed of 100-1,000 rpm, 100-800 rpm, 100-700 rpm, 100-600 rpm, 100-500 rpm or 100-400 rpm.

In certain embodiments, a ratio of the organic phase to the aqueous phase ranges from 1:10 to 20:1. In certain embodiments, the ratio of the organic phase to the aqueous phase is in the range of 1:10-18:1, 1:10-15:1, 1:10-12:1, 1:10-10:1, 1:10-8:1, 1:10-5:1, 1:10-3:1, 1:10-2:1, 1:10-1:1, 1:9-20:1, 1:8-20:1, 1:7-20:1, 1:6-20:1, 1:5-20:1, 1:4-20:1, 1:3-20:1, 1:2-20:1, 1:8-10:1, 1:6-6:1, 1:5-5:1, 1:4-4:1, 1:3-3:1, 1:3-2:1 or 1:3-1:1.

### Step (iv): Removal of Organic Solvent

According to the present application, the removal of the organic solvent may be accomplished under reduced pressure in any suitable manner known in the art, such as rotary evaporation or drying under reduced pressure. In certain embodiments, the organic solvent is removed by rotary evaporation under reduced pressure. In certain embodiments, the rotary evaporation under reduced pressure is conducted at a vacuum degree of less than 0.6 atmosphere (atm), 0.5 atm, 0.4 atm, 0.3 atm, 0.2 atm or 0.1 atm. In certain embodiments, the vacuum degree at which the rotary evaporation under reduced pressure is conducted is in the range of 0.1-0.6 atm, 0.1-0.5 atm, 0.1-0.4 atm, 0.1-0.3 atm or 0.1-0.2 atm.

### Encapsulation Efficiency

A method commonly used in the art may be employed to detere encapsulation efficiency, such as sephadex gel filtration, ultracentrifugation or dialysis. In certain embodiments, dialysis is used to detere encapsulation efficiency.

In certain embodiments, compositions prepared by the method hereof have encapsulation efficiency that is not less than 80%, 83%, 85%, 87%, 89%, 90%, 92%, 93%, 94%, or 95%. In certain embodiments, drug encapsulation efficiency of nanoparticles may reach up to 80%-95%.

### Use in Preparation of Medicament, Method for Treating Disease and Use in Treatment

In one aspect, the present application relates to the use of the pharmaceutical composition hereof in the preparation of a medicament for mitigating, treating, or preventing a disease.

In another aspect, the present application relates to the use of the pharmaceutical composition hereof in the mitigation, treatment or prevention of a disease.

In still another aspect, the present application relates to a method for mitigating, treating, or preventing a disease, comprising applying an effective amount of the pharmaceutical composition hereof on a subject in need thereof.

In certain embodiments, the disease is cancer. “Mitigation”, “treatment” or “prevention” of a disease or condition include preventing or alleviating a condition, slowing the onset or rate of development of a condition, reducing the risk of developing a condition, preventing or delaying the development of symptoms related to a condition, reducing or ending symptoms related to a condition, generating a complete or partial regression of a condition, curing a condition, or some combination thereof.

As used in herein, the term “effective amount” refers to a quantity that can effectuate the treatment of a disease or condition in a subject or can preventively inhibit or prevent the occurrence of a disease or condition. An effective amount relieves to some extent one or more diseases or conditions in a subject, returns to normality, either partially or completely, one or more physiological or biochemical parameters causative of a disease or condition, and/or can lower the likelihood of occurrence of a disease or condition.

The effective dosage of the composition provided herein will depend on various factors known in the art, such as, for example, body weight, age, past medical history, present medications, state of health of the subject and potential for cross-reaction, allergies, sensitivities and adverse side-effects, as well as the adistration route and extent of disease development. Dosages may be reduced or increased by one of ordinary skill in the art (e.g., physician or veterinarian) as indicated by these and other circumstances or requirements.

In certain embodiments, the composition provided herein may be adistered at a therapeutically effective dosage ranging from about 0.01 mg/kg to about 100 g/kg (e.g., about 0.01 mg/kg, about 0.5 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 200 mg/kg, about 500 mg/kg, about 1 g/kg, about 5 g/kg, about 10 g/kg, about 20 g/kg, about 50 g/kg, about 70 g/kg, about 90 g/kg or about 100 g/kg). A given dosage may be adistered at multiple intervals, such as for example once a day, two or more times per day, two or more times per month, once per week, once every two weeks, once every three weeks, once a month, or once every two or more months. In certain embodiments, the adistration dosage may change over the course of treatment. For example, in certain embodiments, the initial adistration dosage may be higher than subsequent adistration dosages. In certain embodiments, the adistration dosage may vary over the course of treatment depending on the response of the subject.

Dosage regimens may be adjusted to provide the optimum desired response (e.g., a therapeutic response). For example, a single dose may be adistered, or several divided doses may be adistered over time.

### SPECIFIC EXAMPLES

The present disclosure will be described below in detail with reference to the following Examples but is not limited to them.

Unless otherwise explicitly indicated, the PEG-PLA copolymers used in the following Examples were obtained from Advanced Polymer Materials Inc. (a Canadian manufacturer of macromolecular materials) and had a molecular weight of 21,000. Additionally, the PEG-PLGA copolymers used in the Examples were also obtained from Advanced Polymer Materials Inc. and had a LG/LA ratio of 75/25 and a molecular weight of 20,000. The dynamic laser scattering instrument used herein was a Zetasizer Nano ZS from Malvern Instruments (UK).

### Example 1

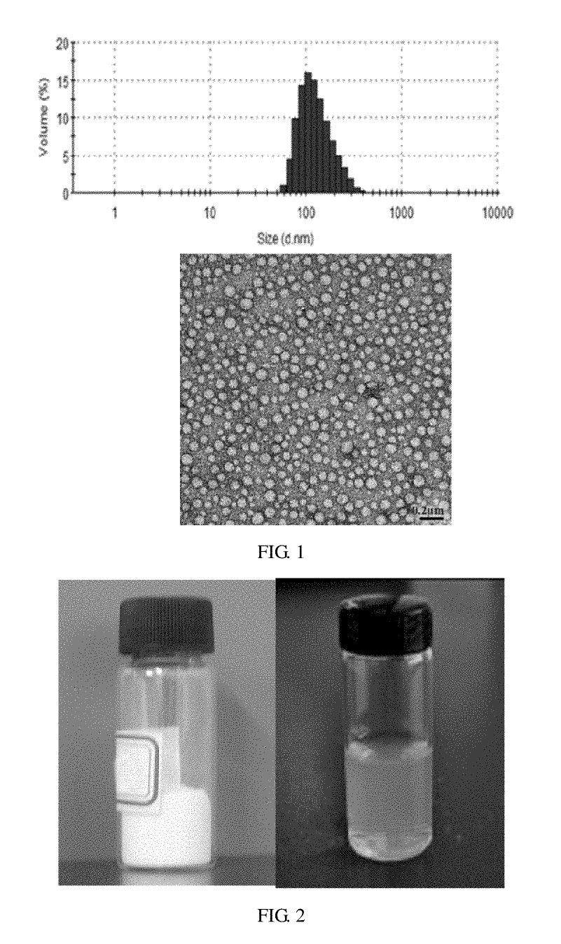

40 mg of PEG-PLA and 2 mg of paclitaxel were co-dissolved under ultrasonic conditions in 5 ml of acetone serving as a solvent. 3 mg of sodium cholate was dissolved in 10 ml of double distilled water. The aqueous solution of sodium cholate was dropped into the acetone solution at a rate of 1 ml/, and the reaction was allowed to run for 10 min at a low stirring speed of 300 rpm. The reaction mass was then transferred into a rotary evaporator, where acetone was removed by rotary evaporation at a vacuum degree of −0.1 MPa for 30 min, resulting in stable paclitaxel-loaded nanoparticles. The nanoparticles were measured on the dynamic laser scattering instrument and detered to have an average particle size of 115.02±11.5 nm and a particle size distribution shown in FIG. 1. They were also detered to have encapsulation efficiency of 91.2±3.5% and a coefficient of dispersion of 0.198.

### Example 2

20 mg of PEG-PLA and 1 mg of docetaxel were co-dissolved under ultrasonic conditions in 5 ml of acetone serving as a solvent. 2 mg of sodium cholate was dissolved in 10 ml of double distilled water. The aqueous solution of sodium cholate was dropped into the acetone solution at a rate of 1 ml/, and the reaction was allowed to run for 10 min at a low stirring speed of 300 rpm. The reaction mass was then transferred into a rotary evaporator, where acetone was removed by rotary evaporation at a vacuum degree of −0.1 MPa for 30 , resulting in stable docetaxel-loaded nanoparticles. The nanoparticles were measured on the dynamic laser scattering instrument and detered to have an average particle size of 89.09±8.9 nm. They were also detered to have encapsulation efficiency of 87.4±4.1% and a coefficient of dispersion of 0.211.

### Example 3

40 mg of PEG-PLGA and 2 mg of cabazitaxel were co-dissolved under ultrasonic conditions in 5 ml of acetone serving as a solvent. 1 mg of sodium cholate was dissolved in 10 ml of double distilled water. The aqueous solution of sodium cholate was dropped into the acetone solution at a rate of 1 ml/min, and the reaction was allowed to run for 10 min at a low stirring speed of 300 rpm. The reaction mass was then transferred into a rotary evaporator, where acetone was removed by rotary evaporation at a vacuum degree of −0.1 MPa for 30 min, resulting in stable cabazitaxel-loaded nanoparticles. The nanoparticles were measured on the dynamic laser scattering instrument and detered to have an average particle size of 78.95±3.3 nm. They were also detered to have encapsulation efficiency of 93.4±2.3% and a coefficient of dispersion of 0.153.

### Example 4

40 mg of PEG-PLA and 2 mg of paclitaxel were co-dissolved under ultrasonic conditions in 5 ml of acetone serving as a solvent. 10 ml of double distilled water was dropped into the acetone solution at a rate of 1 ml/, and the reaction was allowed to run for 10 min at a low stirring speed of 300 rpm. The reaction mass was then transferred into a rotary evaporator, where acetone was removed by rotary evaporation at a vacuum degree of −0.1 MPa for 30 min, resulting in stable paclitaxel-loaded nanoparticles. The nanoparticles were measured on the dynamic laser scattering instrument and detered to have an average particle size of 126.22±14.1 nm. They were also detered to have encapsulation efficiency of 81.2±3.8% and a coefficient of dispersion of 0.178.

### Example 5

### Encapsulation Efficiency of Nanoparticles

High performance liquid chromatography (HPLC) was employed to analyze the amount of paclitaxel. HPLC conditions were as follows: column: Hypersil ODS2 (4.6 mm×250 mm, 5 μm); mobile phase:acetonitrile:water=50:50 (v/v); detection wavelength: 227 nm; flow rate: 1.0 ml/; feed volume: 20 μl. Paclitaxel standard solutions with concentrations ranging from 0.25 μg/ml to 50 μg/ml were analyzed under the above HPLC conditions. A peak area vs. paclitaxel concentration curve was fitted and a regression equation was developed.

For each obtained nanoparticle suspension, a sample thereof was first centrifuged at a low rate of 4,000 rpm for 10 min to get rid of crystals of the drug that were not encapsulated, and then centrifuged at a high rate of 10,000 rpm for 30 min. The supernatant was aspirated away and the remainder was then reconstituted with high-purity water and then dissolved in the same volume of acetonitrile for demulsification. The resulting solution was analyzed under the foregoing HPLC conditions for the amount of paclitaxel contained therein. Meanwhile, an intact sample of the nanoparticle suspension was dissolved in the same volume of acetonitrile for demulsification and measured for the amount of contained paclitaxel under the same HPLC conditions.

Encapsulation efficiency was calculated according to the following equation:

Encapsulation Efficiency(%)=Amount of Drug Encapsulated in Nanoparticles/Total Drug Amount×100%

Encapsulation efficiency of the nanoparticles prepared in Examples 1-3 was averaged at 80-95%.

### Example 6

### Lyophilization of Nanoparticles

Each of the nanoparticle suspensions prepared in Examples 1-3 was centrifuged, added with 10% by volume of sucrose, pre-frozen at −40° C. for 10 hours and freeze-dried in a cold trap at −60° C. for 48 hours, resulting in a long-circulating lyophilized nanoparticle powder. Substantially no change in particle size and no aggregation were observed during the reconstitution of so prepared lyophilized powder.

### Example 7

### In Vitro Stability

Paclitaxel-loaded nanoparticles were additionally prepared by the method of Example 1, respectively with polyethylene glycol (15)-hydroxystearate (HS15) and polyvinyl alcohol (PVA) used in lieu of the cholate. These additional paclitaxel-loaded nanoparticles prepared respectively using HS15 and PVA, together with the paclitaxel-loaded nanoparticles prepared in Example 1, were placed at room temperature. Precipitation of paclitaxel crystals was observed one hour later in the nanoparticles prepared using PVA and two hours later in the nanoparticles prepared using HS15, while no noticeable precipitation of paclitaxel crystals was observed in the paclitaxel-loaded nanoparticles prepared in Example 1 even after it had been placed at room temperature for 3 hours.

### Example 8

### In Vitro Release

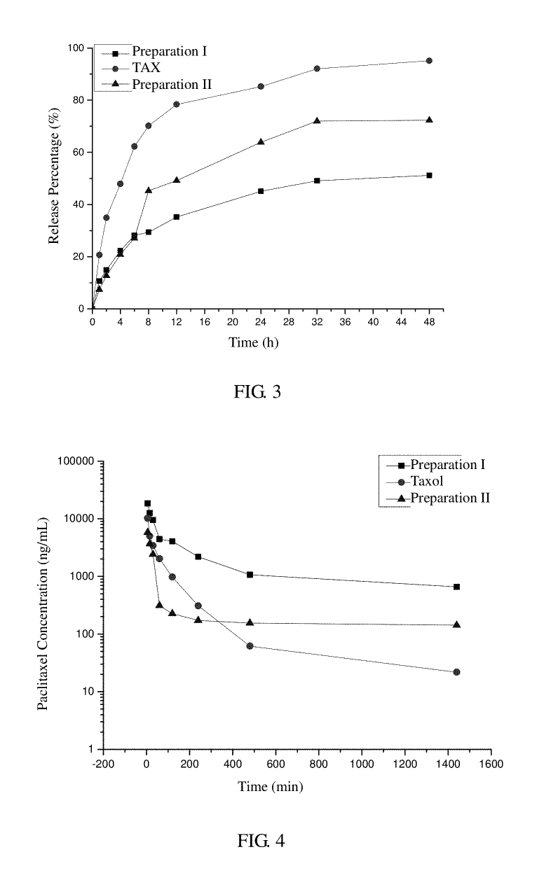

1 mL of the paclitaxel nanoparticles (1 mg/mL) prepared in Example 1, 1 mL of the paclitaxel nanoparticles (1 mg/mL) prepared in Example 4 and 0.167 mL of a commercially available paclitaxel injection (6 mg/mL) were respectively diluted with distilled water to 10 mL. For each of these 10-mL dilutions, 1 ml was reserved as a blank and the remaining 9 ml was filled into a dialysis bag. After the dialysis bag was tightly closed, it was submerged in a 50-ml PBS buffer (pH 7.4, containing 0.2% Tween 80) and shaken on a shaking table at 100 rpm at 37° C. 1-ml samples were taken from the PBS buffer outside the dialysis bag at various times, and each sampling was followed by the addition of fresh buffer to replace the sample volume. Each of the samples was homogeneously mixed with 1.0 ml of acetonitrile and analyzed to detere the amounts of paclitaxel contained therein. Percentages of cumulatively released paclitaxel were calculated, and a release profile was plotted (see FIG. 3). The results showed that, under the in vitro simulated physiological conditions, the release of paclitaxel from the paclitaxel-loaded nanoparticles prepared in Example 1 was much slower than that from the paclitaxel nanoparticles prepared in Example 4 and from the commercially available paclitaxel injection, indicating more excellent sustained-release properties of the nanoparticles resulting from the introduction of the cholate.

### Example 9

### Pharmacokinetics

A. Experimental Animals

Male SD rats weighing 250±20 g were randomly divided into three groups (six in each group), for subsequent use.

B. Test Preparations

Preparation I: the nanoparticles prepared in Example 1;

Preparation II: the nanoparticles prepared in Example 4;

Taxol: a commercially available injection of a concentration of 6 mg/mL of paclitaxel sold under the brand Taxol was used as a control.

C. Adistration and Sample Collection

Preparations I, II and Taxol were each dissolved and diluted to a suitable concentration immediately prior to their use and then given to the respective groups of rats at a dose of 8 mg/kg (counted based on the amount of paclitaxel) via tail vein injection. Blood samples were then collected from the orbital venous plexuses of the rats at different time instants after the adistration into heparin tubes and centrifuged for plasma separation. The plasma samples were stored in an ultra-low temperature freezer at −80° C. for analyses.

D. Plasma Treatment and Deteration

Each of the plasma samples underwent acetonitrile extraction and HPLC analysis to detere the paclitaxel concentrations therein.

E. Results

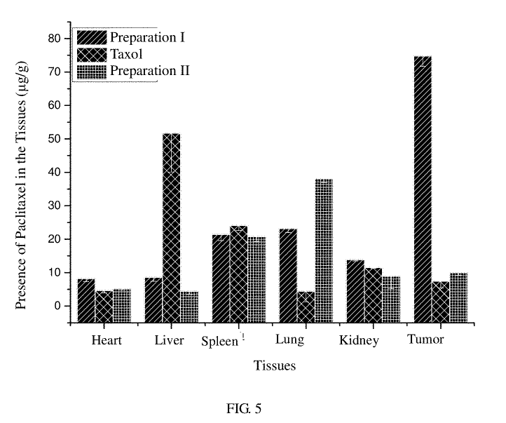

Profiles of the change in the plasma paclitaxel concentration over time are plotted for Preparations I and II (see FIG. 4), and the primary plasma pharmacokinetic parameters were calculated. The results showed that, compared to Taxol and Preparation II, Preparation I resulted in a significantly higher plasma concentration, greater AUC, reduced in vivo eliation rate and prolonged eliation half-life of paclitaxel at the same intravenous adistration dose, reflecting more excellent in vivo release properties of Preparation I than Taxol and Preparation II.

### Example 10

### In Vivo Distribution in Tissues

Fifteen tumor-bearing nude mice were randomly and evenly divided into three groups (i.e., five in each group), and the groups were respectively tail-vein adistered Preparation I, Preparation II and Taxol (10 mg/kg). The mice were killed 2 hours later, and their primary tissues including the hearts, livers, spleens, lungs, kidneys and tumors were removed and accurately weighed. Each of the tissues was added with an amount of normal saline that was three times its weight and homogenized to prepare a tissue homogenate for subsequent use. 500 pL of each tissue homogenate was placed into a 2-mL round-bottom centrifuge tube and then underwent acetonitrile extraction and HPLC analysis under the same conditions as Example 5. Distributions of test preparations in the tissues of the tumor-bearing nude mice are summarized in FIG. 5. As can be seen from the figure, the presence of Preparation I in the tumors is much more than that of Taxol and Preparation II, demonstrating its better targeting performance

### Example 11

### Pharmacodynamics

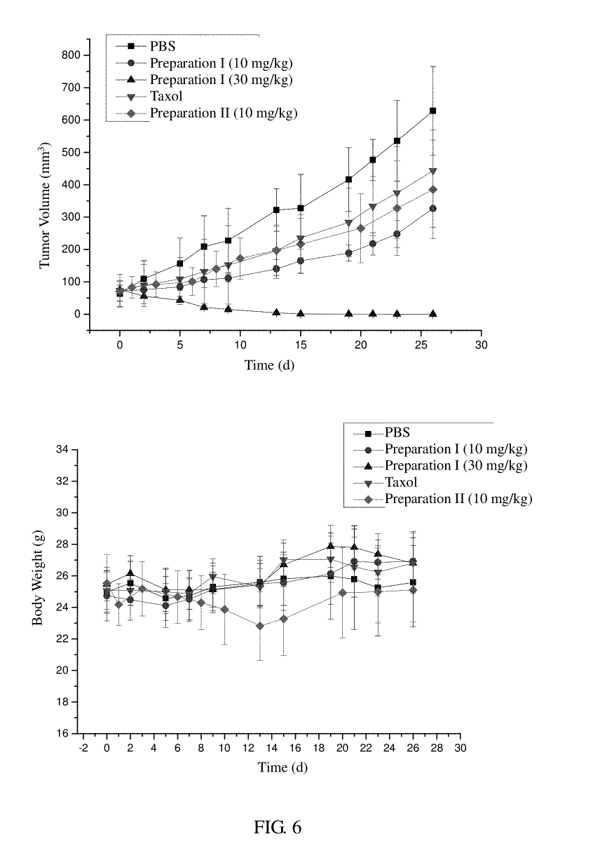

BEL-7402 cells (5×10⁷) were subcutaneously inoculated into the abdomen of male BALB/c nude mice. About two weeks later, tumors in the mice grew to an average volume of 100 mm³ or more. Subsequently, 35 tumor-bearing mice were randomly divided by the tumor volume into the following groups: PBS; Preparation I (10 mg/kg, prepared in Example 1); Preparation I (30 mg/kg, prepared in Example 1); Preparation II (10 mg/kg, prepared in Example 4); and Taxol (10 mg/kg). The adistration was performed intravenously every 3 days for a total of 3 times, and the tumor volumes (volume=ab²/2, where a and b represent the length and width of the tumor, respectively) and body weights of the nude mice were measured every other day following adistration. The results are summarized in FIG. 6, from which it can be seen that Preparation I exhibits a tumor inhibition rate improved over those of Taxol and Preparation II at the same dose. In addition, the tumor inhibition rate of Preparation I shows a significant rise with the adistration dose increased to 30 mg/kg, and some tumors even have disappeared.

1. A pharmaceutical composition, comprising an active ingredient, a polymer and a surfactant, wherein the surfactant comprises a bile salt and the pharmaceutical composition comprises nanoparticles.

2. The pharmaceutical composition of claim 1, wherein the active ingredient is selected paclitaxel, camptothecin or a derivative thereof.

3. The pharmaceutical composition of claim 1, wherein the polymer is selected from PLGA, PLA, a PLGA or PLA derivative or a combination thereof.

4. The pharmaceutical composition of claim 1, wherein the bile salt is selected from sodium cholate, sodium deoxycholate, sodium taurocholate or a combination thereof.

5. The pharmaceutical composition of claim 1, wherein the surfactant excludes lipid surfactants.

6. The pharmaceutical composition of claim 1, which is nanoparticles.

7. The pharmaceutical composition of claim 1, wherein the polymer and the active ingredient are present in a ratio by weight ranging from 5:1 to 40:1.

8. The pharmaceutical composition of claim 1, wherein the surfactant and the active ingredient are present in a ratio by weight ranging from 0.1:1 to 4:1.

9. The pharmaceutical composition of claim 1, wherein the surfactant and the polymer are present in a ratio by weight ranging from 1:5 to 1:50.

10. The pharmaceutical composition of claim 1, which has an average particle size of 50-200 nm.

11. A method for preparing the pharmaceutical composition of claim 1, comprising the steps of:
(i) dissolving the polymer and the active ingredient in an organic solvent;
(ii) dissolving the surfactant in water;
(iii) mixing the aqueous solution from step (ii) with the organic solution from step (i) under the action of a shear force; and
(iv) removing the organic solvent.

12. The method of claim 11, wherein the organic solvent is selected from acetone, dichloromethane, acetonitrile or a combination thereof.

13. The method of claim 11, wherein the shear force in step (iii) results from agitation.

14. The method of claim 11, wherein step (iii) further comprises adding the aqueous solution from step (ii) dropwise to the organic solution from step (i).

15. The method of claim 11, wherein the organic solution and the aqueous solution are present in a ration ranging from 1:10 to 20:1.

16. The method of claim 11, wherein the surfactant is present in the aqueous solution at a concentration of 0.05-1 mg/mL.

17. The method of claim 11, wherein the active ingredient is present in the organic solvent at a concentration of 0.1-1 mg/mL.

18. The method of claim 11, wherein the polymer is present in the organic solvent at a concentration of 2-10 mg/mL.

19. (canceled)

20. A method for relieving, treating, or preventing a disease, comprising adistrating the pharmaceutical composition of claim 1.

21. The method according to claim 20, wherein the disease is a cancer.
